(19) United States
(12) Patent Application Publication
(10) Pub. No.: US 2016/0242623 A1
(43) Pub. Date: Aug. 25, 2016

(54) Apparatus and method for visualizing data and images and for controlling a medical device through a wearable electronic device

(71) Applicants: Alessandro Pasini, Imola (IT); Davide Bianconi, Cesena (IT); Daniele Romani, Imola (IT)

(72) Inventors: Alessandro Pasini, Imola (IT); Davide Bianconi, Cesena (IT); Daniele Romani, Imola (IT)

(73) Assignee: CEFLA SOCIETÁ COOPERATIVA, Imola (IT)

(21) Appl. No.: 15/045,314

(22) Filed: Feb. 17, 2016

(30) Foreign Application Priority Data
Feb. 20, 2015 (IT) BO2015A00088

(51) Int. Cl. A61B 1/00; G06F 3/16; G06F 3/01; G06F 3/0487; G06T 11/60; G06F 3/041; G06F 3/023; A61B 1/24; A61B 6/14; A61C 9/00; A61G 15/02; A61B 6/03; A61B 6/00; A61B 1/04; G10L 15/22

(52) U.S. Cl. CPC A61B 1/00006; G10L 15/22; G06F 3/167; G06F 3/017; G06F 3/0487; G06T 11/60; G06F 3/0416; G06F 3/023; A61B 1/24; A61B 6/145; A61C 9/0046; A61G 15/02; A61B 6/032; A61B 6/4085; A61B 1/04; A61B 1/00048; A61B 1/00039; G10L 2015/223; G06T 2210/41; G06T 2207/30036; A61B 2090/502

(57) ABSTRACT

A dental treatment unit includes n image-generating device, which is connected to a wearable electronic device having an image processor that receives images and displays them on a screen associated to the wearable electronic device. The wearable electronic device enables a visualization of diagnostic images or information of other kind coming from the image-generating device and/or from an operating unit on the screen of the wearable electronic device.

### FIELD OF THE INVENTION

The present invention relates to the field of medical devices, particularly of dentistry. More particularly, the invention relates to an apparatus and a method to visualize images and to control medical devices through a wearable electronic device.

### BACKGROUND OF THE INVENTION

Dental practice is a peculiar environment: on one hand, it can be likened to a surgical environment, in that some operations performed by the dentist interrupt mucosal continuity, and therefore can introduce pathogens (bacteria, virus, fungi) into the tissues of the body under treatment. On the other hand, dental environment is on average much dirtier than most surgical environments. This is due to the particular instrumentation normally used by dentists, which comprises rotary and non rotary instruments (e.g. turbine, micromotor with contra-angle, calculus scaler, etc.), which generate an aerosol cloud containing the bacteria present in the oral cavity. Indicatively, in a milliliter of saliva there are 5 billions of microorganism, some of which can be pathogens or opportunistic.

Details on aerosol generation during dental operation can be found in the chapter “Sterilization, Disinfection and Asepsis in Dentistry” in “Disinfection, Sterilization and Preservation”, Ed. Seymour Block, Fifth Edition, Lippincott, Williams & Wilkins 2001, and also in the Guidelines for Infection Control in Dental Health-Care Settings—2003 Centers for Disease Control Morbidity and Mortality Weekly Report, 2003; 52.

This peculiarity of the dental environment, known since the '70s, induced manufacturers to find ways to control the dental unit without using dentist's hands. A very widespread way is controlling the dental unit (e.g. patient's chair adjustment;, turbine/micromotor increase/decrease of rounds-per-minute, and direction of rotation) through a foot control connected to the dental unit; the foot control being known since the '60s. Nonetheless, using feet to control dental units has some limitations, linked both to the lesser precision of foot controls with respect to hand controls, and to the way of controlling through a foot control, which obliges the dentist to memorize complex sequences of actions (typically a foot control only has a couple of buttons and a lever or joy-stick).

Moreover, since the '90s there have been important innovations in dental imaging field.

On one hand, intra-oral cameras have known a large spread, both to improve dentist-patient communication, and to record the different therapeutic steps for medico-legal reasons. Here, too, given the small dimensions of the camera handpiece, often having just one key, controlling navigation among images or acquired video sequences can become problematic. Often even foot control is difficult to use.

On the other hand, again since the 90's, digital imaging started to spread, first with intra-oral sensors, successively with wider sensor used on panoramic and specific CT apparatuses (extra-oral radiographic apparatuses like panoramic apparatuses and Cone-Beam Computerized Tomography, CBCT). The consultation of radiographs during a dental operation can be of paramount importance, like in e.g. endodontics or metallic implant placement in maxillary or mandibular bone.

An alternative possibility of controlling a device and visualizing images is offered by a recent technology development, wearable electronic devices. At the moment on the market there are wearable electronic devices having approximately the shape of glasses which can be supported by user's nose and ears, in our case by dentist's nose and ears.

Said wearable electronic devices typically comprise:

a portion which can be supported by user's nose;

a portion which can be supported by user's ears;

a housing for electronic circuits, in particular a control module and a memory module;

a camera module;

an output module allowing the user to interact with the wearable electronic device, e.g. a module supplying information to the user in speech form (e.g. a loudspeaker) or visible form (e.g. a display);

a module to show images to the user while she/he is wearing the wearable electronic device;

a module allowing the user to control the wearable electronic device, e.g. a module capable of recognizing speech commands, a module capable of recognizing gestures performed by the user, a module capable of receiving touch commands (e.g. a touch pad);

a module capable of performing a wireless connection (e.g. Bluetooth, WiFi) with other devices in the area around the user.

With respect to image visualization, different kinds of wearable electronic devices are available on the market at the moment, wherein:

Images are visualized on a screen on the edge of lenses,

The screen is part of the lens,

Images are projected directly on the lenses making use of different technologies, e.g. holography.

When in the following description and in claims reference is made to the fact that images are visualized on the wearable electronic device screen, indifferently one of the above-described visualization mode will be used.

With the wearable electronic device dentists are allowed to:

Observe images coming from medical devices in the visible field (intra-oral camera, 3D scanner or other) or from radiographic devices on the wearable electronic device screen, simply glancing up;

Using the wearable electronic device itself to control the medical device he/she is using, be it a dental treatment unit or a radiographic apparatus, and to interact with possible bodies outside the dental practice through remote communication protocols (e.g. consultation with a medical specialist outside the dental practice for telemedicine protocols; medical device's maintenance in contact with a remote specialized technician; link to patient's electronic medical record).

Substantially, on the wearable electronic device screen, information and/or images of different kind can be visualized:

Visible range images: images coming from intra-oral camera, 3D scanner (device digitally acquiring the impression of patient' dental arch), digital camera, periodontal or apical probe, 3D objects renderings, tutorials, educational or entertaining film, intervention protocols;

Images generated by other wavelengths like ultraviolet (UV) or infrared (IR);

Radiographic images: images coming from intra- and extra-oral radiographic apparatuses, e.g. images coming from an intra-oral digital X-ray sensor, allowing the dentist to perform an endodontic intervention;

Information coming from patient's medical record; in this case a link to a dental practice management software must be present;

Information linked to telemedicine: a medical specialist outside the dental practice can follow the intervention and interact with the operator;

Information linked to remote maintenance: a specialized technician in a site outside the dental practice can interact with the dentist to perform a diagnostic intervention on a medical device;

Tutorials and clinical protocols to be consulted during the intervention.

With respect to the visualization of images of different kind, it should be noted that the visualization mode can also be different: in one case, e.g. in the visualization of the patient's clinical record the image could be completely opaque, so hindering the user from seeing her/his environment, while in another case the image could be at least partially transparent, so that the dentist can at the same time visualize e.g. the patient's oral cavity and the radiographic image representing it.

Each image, according to the kind of image, the device that generated it, the technology through which the image itself is transferred to the wearable electronic device, and the mode through which the image is visualized by the wearable electronic device, can be processed through a more or less complex chain of components. These components can be distributed among the various devices and/or be integrated in few (to the limit one) main image processing units: the set of said components is called image processor.

To control the medical device (dental treatment unit or radiographic apparatus) through the wearable electronic device, the dentist can use different technologies, among which (including but not limited to):

Speech recognition through a microphone inside the wearable electronic device;

Gesture recognition through a camera inside the wearable electronic device;

Eye tracking through a camera inside the wearable electronic device;

Manual input devices, with keys or touch surface inside the wearable electronic device.

Typically, the communication between wearable electronic device and medical device to be controlled occurs through wireless communication protocols like e.g. Bluetooth, WiFi, WiFi Direct.

The command which can be provided to a dental treatment unit are (including but not limited to):
a) Adjustment of patient chair (e.g. seat height and backrest tilting);
b) Adjustment of rotary and non-rotary dental instruments on the dentist's instrument board (e.g. number of rounds per minute and direction of rotation for rotary instruments);
c) Control of dental radiographic apparatuses;
d) Acquisition (e.g. freezing of video images) and adjustment of parameters (e.g. brightness, magnification, colors) of the images coming from a dental camera;
e) Visualization of multimedia contents by the dentist, among which navigation in the image archive from the camera or already acquired radiographs;
f) Personal data, treatment plan, already performed therapies, information from the patient's digital record visualized on the screen;
g) Visualization of multimedia contents on the screen by the patient;
h) Switching on and off, light emission parameters adjustment of the operating lamp;
i) Reproduction of the controls of the keypad or console;
j) Dental treatment unit maintenance;
k) Control of dental treatment unit accessories: glass, suction;
l) Control of apparatuses outside the dental treatment unit and linked to it (e.g. doorphone);
m) Recognition/authentication of operator and/or patient, e.g. through bar codes, QR codes, RFID, face detection;
n) Start of cleaning/disinfection/sterilization cycles in specific apparatuses, or reception of the information that a cycle is completed.

The commands that can be provided on a radiographic apparatus are (including but not limited to):
a) Adjustment of the apparatus in order to fit it to a single patient (e.g. exposure parameters, height of the apparatus);
b) Moving mechanical parts in order to hold parts of patient's body in the position desired for the acquisition: often operator's hands are both engaged during patient's positioning;
c) Adjustment of laser guides for patient positioning;
d) Emergency procedure to stop X-ray emission;
e) Setting of the desired acquisition protocol;
f) Emission of X-rays once the patient has been correctly positioned.

Each command, according to the kind of command, input technologies in the wearable electronic device, the medical device to which it has to be delivered, and mode through which the command is transferred from the wearable electronic device to the medical device to be controlled, can be processed through a more or less complex chain of components. These components can be distributed among the various devices and/or integrated in few (to the limit one main control units: the set of said components is called controller.

### SUMMARY OF THE INVENTION

All that has been said above makes the possibility very interesting, on the one hand to control the dental treatment unit, but also imaging apparatuses, without contact with the dentist's hands, in that the dentist's hands during operation are typically contaminated in the best case with the patient's saliva, in the worst case with blood. On the other hand, the possibility of visualizing the images acquired through intra-oral cameras, X-ray digital sensor, or an extra-oral radiographic apparatus on the screen of a wearable electronic device is very interesting for the dentist, without the need to use her/his hands to navigate from an image to another, visualizing them instead on the virtual screen of a wearable electronic device.

This object is achieved by an apparatus and a method according to the invention. Advantageous embodiment and refinements are specified in the claims dependent thereon.

The advantages of the present invention are essentially the possibility to control without contaminating the medical device in use (dental treatment unit or radiographic apparatus), and in the possibility of visualizing a plurality of images easily going from one to another, without distracting dentist's look from her/his operating field.

Known dental treatment units can be controlled by the dentist through foot control, but in this case she/he has to memorize complex control sequences, or she/he can use her/his hands to press keys present on the dentist's instrument board or the touch screen of console or screen, but in this second case the dentist contaminates the dental treatment unit with her/his hands soiled with saliva and/or blood. Dental treatment units controlled through speech recognition are known in the art, but these have the disadvantage that the dentist has to move her/his gaze from the operating field to visualize the desired image.

For all said above, it is apparent that the dental treatment unit is the preferred embodiment of the present invention. Nonetheless, the skilled person can apply the same concepts to other kinds of apparatuses, in particular radiographic apparatuses, in the dental practice, or more generally, in a medical office.

Since the dental treatment unit is the main work tool for the dentist, the dental treatment unit is conceived as a “hub” to which all the other important devices in the dental practice make reference, like e.g.:

An intra-oral radiographic apparatus in combination with an X-ray digital sensor, a panoramic radiographic apparatus, a volumetric radiographic apparatus (CBCT),

Devices in the instrument processing room (e.g. ultrasonic cleaner, thermal disinfector, autoclave).

The dental treatment unit is the preferred embodiment for the present invention. Nonetheless, the same concepts are easily applicable by the skilled person to any other medical device.

In the first case, the dentist can visualize through the wearable electronic device all the radiographic images acquired through these apparatuses. In the second case on the dental treatment unit and therefore on the wearable electronic device information on the cycle status of the cleaning/disinfecting/sterilizing apparatus are received (e.g. the information that a cleaning/disinfecting/sterilizing cycle is finished).

### BRIEF DESCRIPTION OF THE DRAWINGS

Further advantages and properties of the present invention are disclosed in the following description, in which exemplary embodiments of the present invention are explained in detail based on the drawings:

FIG. 1: Schematic representation of medical devices and images inside a dental practice;

FIG. 2: Dental treatment unit schematic representation;

FIG. 3: Detail of a dentist's instrument board with an X-ray intraoral sensor;

FIG. 4: Simplified schematic representation of a graphical interface;

FIG. 5: Workflow of a preferred embodiment.

### DETAILED DESCRIPTION OF EMBODIMENTS OF THE INVENTION

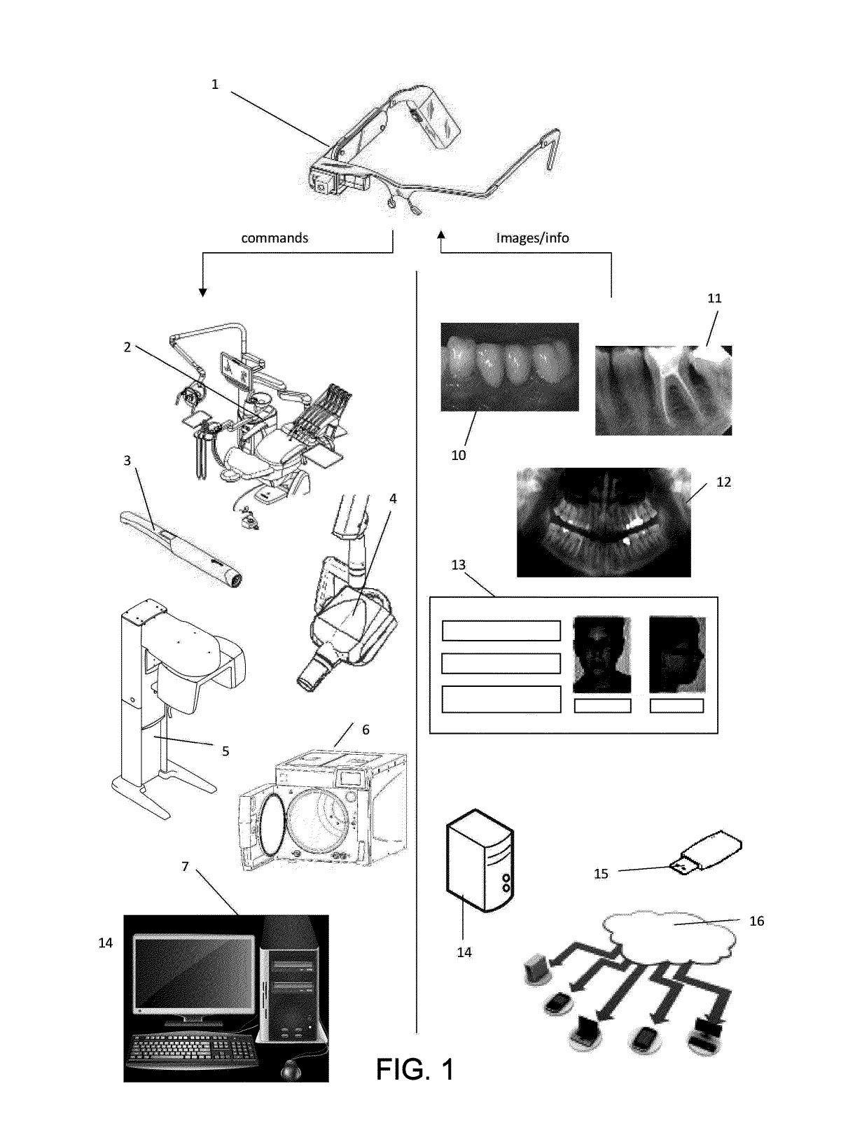

FIG. 1 shows a schematic representation of the interconnections of the wearable electronic device with the different medical devices and the different kinds of images within the present invention.

On the left side, the typical medical devices that can be controlled by the wearable electronic device 1 are shown: dental treatment unit 2, intra-oral camera 3, intra-oral radiographic apparatus 4, extra-oral radiographic apparatus 5, cleaning/disinfecting/sterilizing devices 6 for dental instruments, workstation 7.

On the right side, the images which can be typically visualized on the wearable electronic device screen are shown:

Static images 10 of the visible field, e.g. images coming from an intra-oral camera, 3D scanner, dental cameras, periodontal or apical probes, 3D objects rendering;

Dynamic images of the visible field (not shown) like e.g. streaming videos coming from an intra-oral camera, tutorials, educational or entertaining films, intervention protocols, learning protocols;

Images generated through other wavelengths like ultraviolet and/or infrared (not shown)

Radiographic images: images 11 coming from intra-oral radiographic devices;

Radiographic images 12 coming from extra-oral radiographic devices, e.g. from a panoramic apparatus or a Cone-Beam Computerized Tomograph (CBCT);

Information coming from patient's digital record 13; in this case a link to a dental practice management software must be present;

Images coming from archives 14, removable devices 15 (e.g. USB stick) and from remote archives 16 (cloud computer);

Information linked to remote assistance (not shown): possibility for a specialized technician in a site outside the dental practice to interact with the dentist in order to perform a diagnostic intervention on a medical device.

It should be finally noted that wearable electronic devices can also generate images, in the form of photographs, or clips, therefore also these images can be saved in the patient's electronic record and visualized successively.

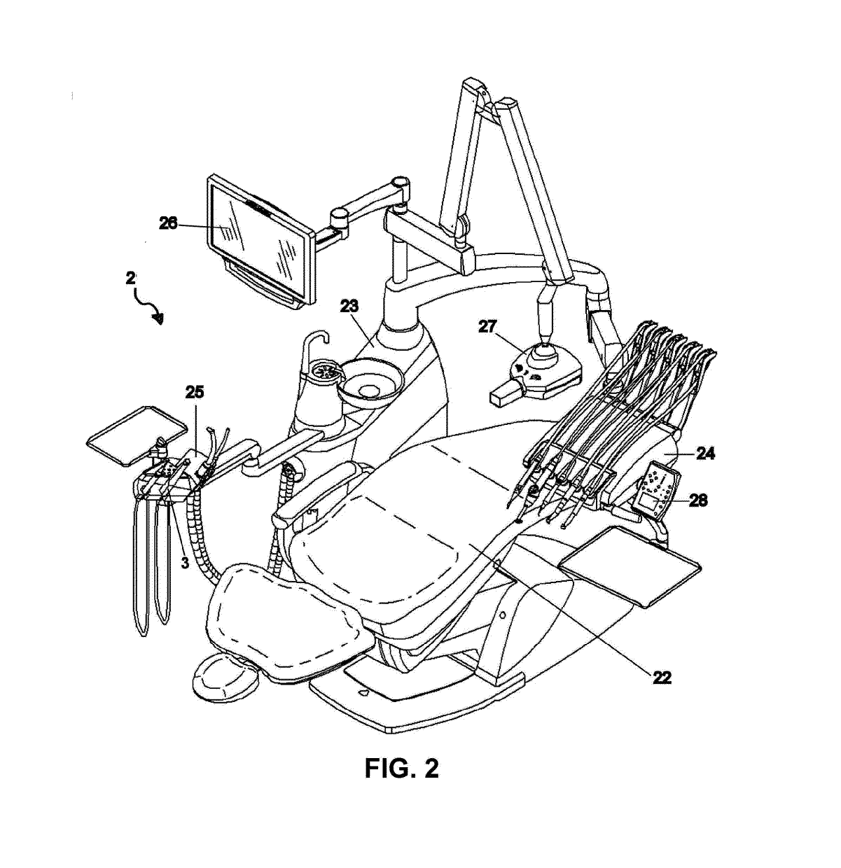

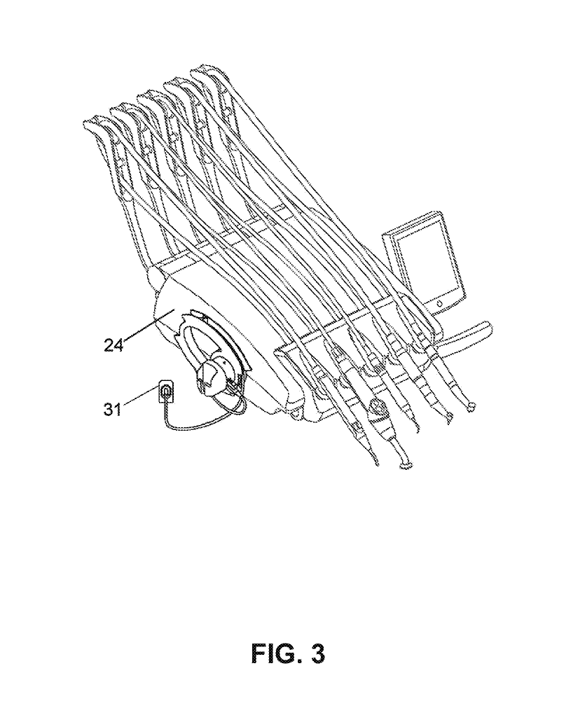

FIG. 2 shows a typical dental treatment unit of the known art, indicated on the whole with 2, comprising the different parts typically forming it. In FIG. 2 there are shown a chair 22, a hydrogroup 23, a dentist's instrument board 24, an assistant's instrument board 25, a monitor 26, which can be connected or not to an external personal computer (PC) (not shown), an intra-oral X-ray unit 27 supported by an arm linked to the hydrogroup 23. Moreover, the dental treatment unit may comprise an operating lamp (not shown) and an X-ray digital sensor 31 (visible in FIG. 3).

On the dentist's instrument board 24 the typical instruments used during dental therapies can be recognized: an air/water dental syringe, a curing lamp, an ultrasound scaler for removing calculus, a micromotor with a contrangle, a turbine. On the assistant's instrument board 25 a camera is present, whose images can be visualized in real time on monitor 26. If the dental treatment unit 2 is connected to an external PC or a workstation (not shown), the digital patient record can be consulted, comprising all patient's information like personal data, therapy plan, already performed therapies, already acquired visible or X-ray images. Moreover, on dentist's instrument board 24 a dentist's control console 28 is typically present, which allows to modify the operating parameters of dental unit 2. The control console 28 is typically provided with a small display for visualizing information. On the most advanced versions of the control console 28 or on the screen 26 different kinds of information can be visualized, among which information on the patient, on the already performed therapy or patient's radiographic images.

FIG. 3 shows a detail of a dentist's instrument board, which supports an X-ray digital sensor 31, to be used in connection with the intra-oral radiographic apparatus 27.

It is apparent that all the instruments need controls in order to be used, starting from patient's chair 22 adjustment. Nowadays most instruments are controlled through foot control, with more or less complex combinations of sequential actions. Often, to make controlling more user-friendly, the removal of an instrument from instrument board 24 leads to showing on control console 28 the menu relative to the adjustment of the instrument in use in that moment.

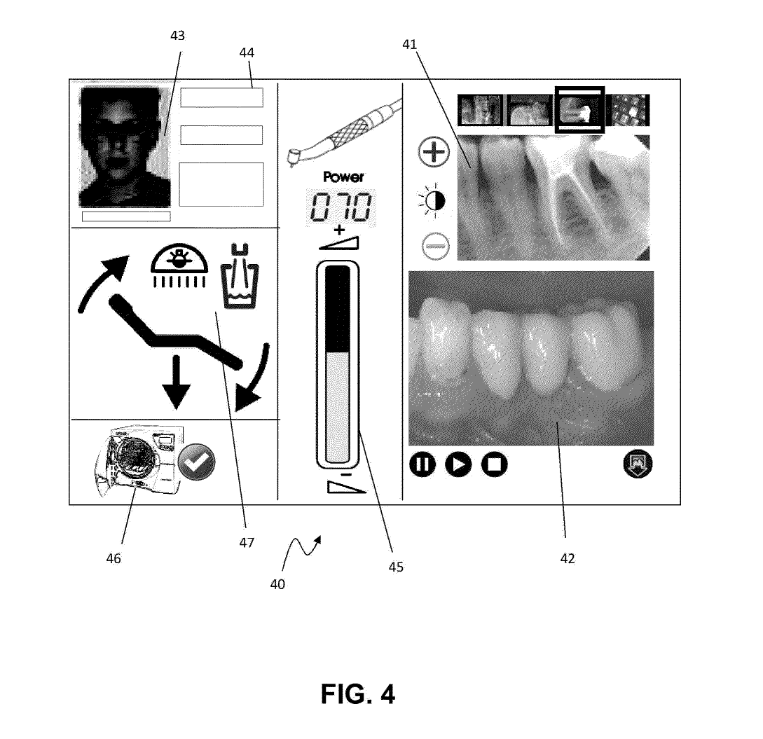

FIG. 4 shows a graphical interface 40, which can be visualized on the screen 26 of the dental treatment unit, or on the display 28 of the dentist's instrument board 24, or on the screen of workstation 7. Said graphical interface shows e.g. a radiographic image 41, a streaming video 42 generated by the intra-oral camera, a picture 43 of the patient with her/his personal and clinical data 44, an adjustment bar 45 for adjusting the instrument in use, the status 46 of the cleaning/disinfecting/sterilizing devices, controls 47 for adjusting patient's chair.

Concerning adjustment bar 45 of the instrument in use, it should be noted that the use (i.e. its removal from the instrument board) of the instrument (e.g. water/air dental syringe, curing lamp, calculus ultrasonic scaler, micromotor with contrangle, turbine, intra-oral camera) causes the appearance of an adjustment bar specific for that specific instrument. For instance, when the micromotor is in use, an adjustment bar will appear allowing to choose the number of rounds per minute and the direction of rotation of the micromotor, while when the intra-oral camera is in use, an adjustment bar will appear allowing to choose whether to acquire a clip or a frozen single image.

In the present invention the graphical interface 40, which is traditionally visualized on the above-said screens 26, 28, or 7, is moreover visualized on the wearable electronic device screen. A specific pre-set speech control can be associated to each control of the graphical interface 40, so that the operator can control the devices neither using her/his hands, nor lifting her/his gaze from the operating field.

The speech controls are acquired by the wearable electronic device, processed and translated into electronic signals allowing to control medical devices

Studying a graphical interface 40 suitable to easily control all the parameters listed in paragraph 0017 is in the normal abilities of the skilled person.

An alternative possibility is that the wearable electronic device directly controls the medical device, without passing through the graphical interface 40; in this case pre-set commands, e.g. speech commands, are directly translated into electronic signals allowing to control the medical devices connected to it. Advantageously the communication between wearable electronic device and medical device to be controlled occurs through wireless communication protocols, e.g. Bluetooth, WiFi, WiFi Direct.

It should also be specified that the connection between wearable electronic device 1 and dental treatment unit 2 can occur in two alternative ways:

The connection between wearable electronic device and dental unit can be direct and local;

The connection between wearable electronic device and dental unit can be indirect and occur through a remote server. This second possibility appears particularly interesting in the case of a dental practice provided with a plurality of dental treatment units, and wherein the management of patients and appointments occurs through a management software for the dental practice.

According to an improvement of the invention, the wearable electronic device can be used as a magnifying device of dentist's visual field. In particular, in this combination, the electronic wearable device can visualize video images of the operating field, either previously acquired or directly real time, through at least a camera shooting the operating field. The acquired image can be magnified as desired through commands provided to the image processing electronics and/or of the wearable electronic device and visualized in the said device according to one or more of the previously described modes. During operation, dentist is allowed to pass from a direct vision in a 1:1 scale, or, if she/he has to be extremely precise, she/he can replace her/his direct vision with a real time, but magnified image, of the intervention area.

Said image can be visualized in different areas of the screen of the wearable electronic device or make said image a replacement of the direct visual image.

The above-described application converts the wearable electronic device into a sort of digital magnifying lens.

According to a further improvement, the wearable electronic device can be in combination with visualizing means of previously acquired diagnostic images, e.g. 3D images, identifying means on said 3D diagnostic images of univocal points for the definition of a fixed spatial reference system, said points corresponding to given markers that can even be purely anatomic. A processing section detects the anatomic markers on the patients, registers the video images to the previously acquired diagnostic 3D image, and transmits and visualizes the previously acquired image of the registered 3D volume to the visual image on the lens of the wearable electronic device, in a combined condition with the visual condition.

The combination can occur using visual images shot through a camera, and therefore visualizing a digital fusion image replacing the direct vision, or the combination can occur visualizing the image data of the previously acquired diagnostic three-dimensional image with a given transparency on the screen of the wearable electronic device, so that a natural fusion can occur between direct visual image and previously acquired diagnostic image.

A further embodiment can have tracking means of the patient's position and of a surgical instrument with respect to a fixed reference system and the visualization in fusion images even of the active part of the instrument, like e.g. the tip of a turbine or an endodontic file.

Finally, it should be pointed out that the image processor of the images generated by one of the devices capable to generate images can be:

Totally inside the dental treatment unit (2) or inside another medical device, or

Totally inside the wearable electronic device (1) or

At least a part of the operative components of the image processor receiving the external images transmitted by one or more devices capable of generating images can be inside the wearable electronic device (1), while the remaining part of the operative components of the image processor is inside said devices or in a centralized image processing unit and connected to said devices and with said wearable electronic device.

In a preferred embodiment, the wearable electronic device 1 is used to visualize the images that are generated by medical devices connected with the dental treatment unit 1, like the intra-oral camera 3 and the intra-oral X-ray digital sensor 31. In a further preferred embodiment, the wearable electronic device is used to visualize patient's digital record 13. Therefore, the dental treatment unit works as a hub.

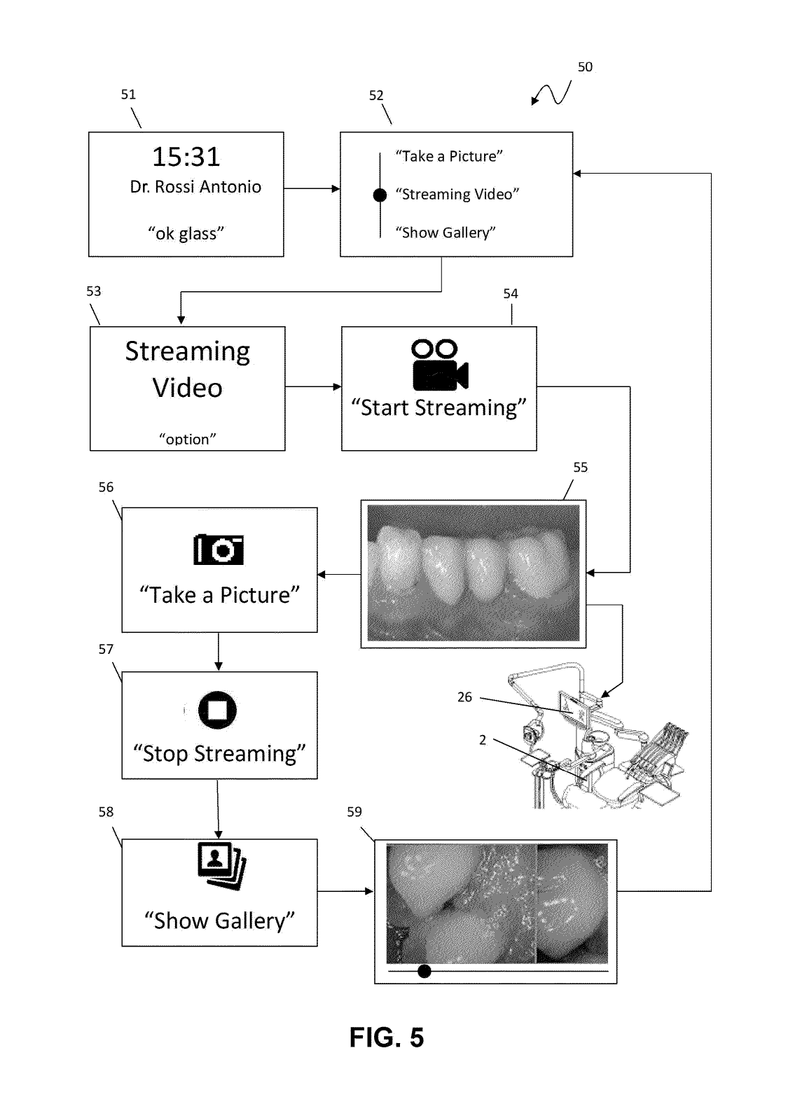

In this embodiment, shown in FIG. 5, the dentist puts on the wearable electronic device 1, and on the wearable electronic device's screen appears an initial menu 51, which the dentist can activate through the command “OK glass” (speech command) or using her/his finger to tap on the wearable electronic device itself (touch command). The following screen 52 shows a menu from which the dentist can choose an application like “take a picture”, “streaming video”, “show gallery”. Now, for instance, to take a picture of the patient in front of her/him, the dentist can pronounce the words “take a picture” or can use a touch command in order to activate the camera inside the wearable electronic device itself and thus shoot a photograph. This photograph can successively be shown inside a gallery 59 of the images on the wearable electronic device 1 screen and be steadily saved in patient's digital record 13.

Alternatively, the dentist can choose the option “streaming video” of the intra-oral camera 3: this activates screen 53, from which, through a speech or touch command, screen 54 appears, showing the signal picked up by camera 3 on the screen of the wearable electronic device 1. At this point, the dentist frames with the camera the anatomical portion of interest, which she/he can see on screen 55 without diverting her/his gaze on screen 26, which is instead turned towards the patient, in order to facilitate dentist-patient communication. Once the dentist finds the frame of interest, she/he can, using a speech or a touch command, freeze an image 56 of the streaming video and save it through the command “take a picture”. Once the desired number of images has been saved, the dentist can stop the streaming video 57 of the intra-oral camera 3 through speech or touch command on the wearable electronic device. At this point, again through the speech or touch command “show pictures” 58, the dentist can access the gallery 59, in which all the acquired images can be visualized. If the dental treatment unit 2 is connected to dental practice management software, in the gallery 59 images saved in preceding sessions can be visualized, too, and the new images of the gallery are permanently saved in the patient's digital record 13.

An alternative working mode to the above-described one consists in the fact that the commands “start streaming” 54, “take a picture” 56, “stop streaming” 57, “show picture” 58 are performed not through the speech or touch commands of the wearable electronic device 1, but through the traditional commands of the dental treatment unit 2. Therefore, in the example of the workflow shown in FIG. 5, the removal of the intra-oral camera 3 from its seat in the dental treatment unit 2 starts the streaming video 54, while at the same time the video is shown on the screen 26 of the dental treatment unit 2 and on the screen of the wearable electronic device 1. The freezing of the image 56 is performed through a key on the camera handpiece 3 or through the foot control (not shown) of the dental treatment unit 2. The re-positioning of the camera handpiece 3 inside its seat in the dental treatment unit 2 is the equivalent of the command stop streaming 57.

The wearable electronic devices 1 possess a general-purpose logic, and therefore are based on known communication standards, e.g. TCP/IP. The challenge for the skilled person is to ensure the cooperation between the wearable electronic device 1 and a dental treatment unit 2, which does not have those functionalities, providing it with an efficient communication interface allowing them to interact smoothly.

While the invention has been described in connection with the above described embodiments, it is not intended to limit the scope of the invention to the particular forms set forth, but on the contrary, it is intended to cover such alternatives, modifications, and equivalents as may be included within the scope of the invention. Further, the scope of the present invention fully encompasses other embodiments that may become obvious to those skilled in the art and the scope of the present invention is limited only by the appended claims.

The invention claimed is:

1. A dental treatment unit comprising one or more image-generating devices selected from the group consisting of an intra-oral camera, a 3D dental scanner, apical and/or periodontal probes, a videoradiographic intra-oral or extra-oral X-ray sensor, a generator of graphical interfaces of a control unit of the dental treatment unit, and control units of one or more additional independent operating units that are web-connected with a control unit of the dental treatment unit; a wearable electronic device; and a controller operatively coupled thereto, the wearable electronic device comprising:
an image processor receiving external images transmitted from the one or more image-generating devices and displaying the external images on a screen operatively coupled to the wearable electronic device, some operative components of the image-generating devices being located inside the image-generating devices or in a centralized processing device and being connected with the image-generating devices and with the wearable electronic device; and
a control signal input unit comprising one or more of the following units:
a processor of audio signals, said processor of audio signals converting a speech command by an operator into a pre-set command for the dental treatment unit and optionally for one or more of said image-generating devices and one or more of the additional independent operating units connected in a web with the dental treatment unit;
a sensor for gesture recognition, said sensor for gesture recognition converting a signal received in form of a gesture into a pre-set command for the dental treatment unit and optionally for one or more of the image-generating devices and one or more of the additional independent operating units connected in a web with the dental treatment unit; and
a manual input device, wherein a touch by an operator on a graphical interface produces a pre-set command for the dental treatment unit and optionally for one or more of said image-generating devices and one or more of the additional independent operating units connected in a web with the dental treatment unit,
wherein the wearable electronic device is adapted to allow visualizing diagnostic images or information of other kind coming from one or more of the image-generating devices and/or from the one or more additional independent operating units on the screen of the wearable electronic device.

2. The dental treatment unit according to claim 1, wherein the control signal input unit is adapted to receive control commands for operating and/or adjusting the dental treatment unit from the operator through one or more of the input units providing the signals to the controller associated to the wearable electronic device, which converts said signals into control signals for the dental treatment unit.

3. The dental treatment unit according to claim 2, wherein the control commands are actuated through pre-set speech commands.

4. The dental treatment unit according to claim 1, wherein a graphical interface is visualized on a second screen and replicated on the screen of the wearable electronic device, and wherein the graphical interface enables control of the dental treatment unit operatively coupled to the graphical interface.

5. The dental treatment unit according to claim 1, wherein one or more commands received through the wearable electronic device are selected from the list consisting of the following commands:
adjustment of patient chair, or seat height and backrest tilting;
adjustment of rotary and non-rotary dental instruments on a dentist's instrument board, and a number of rounds per minute and direction of rotation for rotary instruments;
control of dental radiographic apparatuses including intraoral radiographic apparatus, panoramic apparatus, and volumetric CBCT;
acquisition and freezing of video images and adjustment of parameters of the images coming from the intra-oral camera;
visualization of multimedia contents by the dentist, comprising navigation in an image archive from the intra-oral camera or already acquired radiographs;
personal data, treatment plan, already performed therapies, information from a patient's digital record visualized on the screen;
visualization of multimedia contents on the screen by the patient;
switching on and off, light emission parameters adjustment of an operating lamp;
reproduction of controls of a keypad or console;
dental treatment unit maintenance;
control of dental treatment unit accessories, dental unit glass or suction unit;
control of apparatuses outside the dental treatment unit and linked to the dental treatment unit;
recognition/authentication of the operator and/or the patient;
start of cleaning/disinfection/sterilization cycles in specific apparatuses, or reception of information that a cycle is completed;
real time generation and visualization of magnified visual images of an operating field, replacing or in combination with direct visual images, and/or a control of a magnifying scale;
real time generation and visualization of fusion images of previously acquired diagnostic images with direct visual images through registering of two images; and
visualization of active parts of instruments through tracking and digital reproduction of icons representing the active parts superposed to a visualized anatomic or component image.

6. The dental treatment unit according to claim 1, wherein the wearable electronic device is configured as an object balanced on the operator's nose and ears, and wherein:
images are visualized on a screen on edges of lenses,
the screen is part of a lens, or
images are projected directly on the lenses making use of mage-reproducing technologies, the image-reproducing technologies comprising holography.

7. A method of using a dental treatment unit comprising one or more image-generating devices selected from the group consisting of an intra-oral camera, a 3D scanner, apical and/or periodontal probes, a videoradiographic intra-oral or extra-oral X-ray sensor, a generator of graphical interfaces of a control unit of the dental treatment unit and optionally control units of one or more independent operating units connected in a web with a control unit of the dental treatment unit, and further optionally comprising a connection to a dental practice management software or a connection to remote archives, and further comprising a wearable electronic device comprising:
at least a part of operative components of an image processor receiving external images transmitted from one or more of the one or more image-generating devices and showing the external images on a screen associated to the wearable electronic device, the remaining part of the operative components being located inside the image-generating devices or in a centralized processing device and connected with the image-generating devices and with the wearable electronic device;
a control signal input unit comprising one or more of the following units:
a processor of audio signals, the processor of audio signals converting a speech command by an operator into a pre-set command for the dental treatment unit and optionally for one or more of the image-generating devices and one or more of said units connected in a web with the dental treatment unit;
a sensor for gesture recognition, said gesture recognition sensor converting a signal received in the form of gesture in a pre-set command for the dental treatment unit and optionally for one or more of said devices capable of generating images and one or more of the independent operating units connected in a web with the dental treatment unit; and
a manual input device, wherein a touch by the operator on a graphical interface produces the pre-set command for the dental treatment unit and optionally for one or more of the image-generating devices and one or more of the independent operating units connected in a web with the dental treatment unit,
wherein the wearable electronic device enables visualizing diagnostic images or information of other kind on a screen of the wearable electronic device without moving the operator's view from an operating field.

8. The method according to claim 7, wherein controls of operation and/or adjustment of the dental treatment unit are actuated by the operator through one or more of the input units providing signals to the controller associated to the wearable electronic device which converts the signals into control signals for the dental treatment unit.

9. The method according to claim 7, wherein the wearable electronic device is connected to the dental treatment unit through a graphical interface.

10. The method according to claim 7, wherein the wearable electronic device is directly connected to the dental treatment unit.

11. The method according to claim 7, wherein the information visualized on the screen of the wearable electronic device is one or more of:
images in a visible field coming from the intra-oral camera, 3D scanner, or periodontal probe;
radiographic images coming from intra-oral or extra-oral radiographic apparatuses;
images coming from both local and remote archives;
streaming video coming from intra-oral or extra-oral cameras, tutorials, or educational films;
the audio signals;
patient's digital medical record;
magnified visual images of an intervention area in replacement of or in combination with direct visual images;
control of a magnifying scale; and
images of previously acquired diagnostic images fused with direct visual images through registration of the two images.

12. A medical device comprising one or more image-generating devices and a controller coupled to a wearable electronic device, the wearable electronic device comprising:
at least a portion of operative components of an image processor receiving external images transmitted from one or more of the image-generating devices and showing the external images on a screen associated to the wearable electronic device, a remaining portion of the operative components being inside said image-generating devices or in a centralized processing device and connected with the image-generating devices and with the wearable electronic device;
a control signal input unit comprising one or more of the following units:
a processor of audio signals, the processor of audio signals converting a speech command by an operator in a pre-set command for the medical device and optionally for one or more of the image-generating devices and one or more of the units connected in a web with the medical device;
a sensor for gesture recognition, said gesture recognition sensor converting a signal received in the form of gesture in a pre-set command for the medical device and optionally for one or more of said devices capable of generating images and one or more of the units connected in a web with the medical device; and
a manual input device, wherein a touch by the operator on a graphical interface produces a pre-set command for the medical device and optionally for one or more of the image-generating devices and one or more of the units connected in a web with the medical device,
wherein the wearable electronic device enables visualizing diagnostic images or information of other kind on a screen of the wearable electronic device without moving the operator's view from an operating field.

13. The medical device according to claim 12, wherein controls for operating and/or adjusting the medical device are actuated by the operator through one or more of input units providing signals to the controller associated to the wearable electronic device, the controller converting the signals in control signals for the medical device.
